SEQUENCE LISTING (22 sequences)

SEQ ID NO: 1 - DNA, 447 bp, synthetic construct
  source 1..447: mol_type = other DNA
atggagactgtctccgccgttaaccaaacactccccatctccggcggtgaaccggtgaagttcacgacatactctgccgcagttcacaaagtccttgtcatggtcaacgccggaattctagggttactccagcttgtcagccaacagtcctctgttttggaaacacataaagccgcattcctttgtttctgtgttttcattctattctacgccgttcttcgcgtccgtgaagccatggacgtgcgcctacaacccggtttagtcccaaggctcattggacatgggagccatctcttcggtggtctagccgctcttgtgctcgtctccgtcgttagtaccgcgttttctattgtcctttttcttctctggtttatctggctctccgccgtcgtctacttagagacaaacaaacccagtgcctgtccacctcagttaccaccagtttaa

SEQ ID NO: 2 - protein, 148 aa, synthetic construct
METVSAVNQTLPISGGEPVKFTTYSAAVHKVLVMVNAGILGLLQLVSQQSSVLETHKAAFLCFCVFILFYAVLRVREAMDVRLQPGLVPRLIGHGSHLFGGLAALVLVSVVSTAFSIVLFLLWFIWLSAVVYLETNKPSACPPQLPPV

SEQ ID NO: 3 - DNA, 4800 bp, synthetic construct
  source 1..4800: mol_type = other DNA
gatctggtaatgcgctgattctatcgcactccattatcgctacaaaaacaaggacaatatcaacaagaagaacgaaccatatgccttgagagtttctcaattaattccaaaaactagggtttattaaagtcggttaaaaaacaacatgagctagttctatagcactctatcgcttaaagataaagaaaaacaacgacaaaataaaccaacaaagtcgaatcatatgccttgagagtttgttgagtatacttacaatcgagggaggaggaatgaagaacgtaactaaccgacggtcaagtgaatatggccggagatctagctttaaggtgagcaagtgatacctactatatagtttatatagtttttcataaagacctttacaaggataacatttacaaaaacatttaccattataattaaatacattagctttttaatatatttcttaattttattaattatacatagatatgttttctattttattgagaacaaaatcttctcaaaatctagttgtcaaattttcattcaacttttaattataaaaaataagaaatttaaaagtaacttgtattcaaaaatgtcaaattaacaaaaaaattgtggtctcacacattattttctaactgctattcttaaattcgcaactaatatttgcttaatcaaatttactttcattttgaaataattatattttatccaaaaaaaggactattgtaaaataaatcaagattttttaaactaatattataatatatcaagatatttttatataattccattaaaaatctataatccccatcaaaattgattctatcatttgattattatttattaagattatatatcttattatataaatcttagttttcaaatatgtaaggaatataatttaacatatattaagtactcaataaaatttagttgcaaatatttatgtgtttatgatatcaacatattcaactaaacaattaataaggaggtaacatgtgttttaaaaattgtatgaaatgtgaaagtttgctcactcatattttgatcatccagttccagctaaactaaagaagttaaggtcccataaccgagaagtttgtcaaaagtttttattagatatttttaaatttaagaaacagataaactttacatggatttatcaatatcttcagatactttagataagtccattgaccacggcaaaggattattttaaagtacgtgaaaaaacattaagaaacagaaacaaaaagaaaaatgaaaagtttcgggaatgtttggatatcaagtaaataaaatgaattcgaagccttctctctttagaacttcgagtttttcgtctgataacgatgtttacgtattattatgatcatctcgcggctgcggagaaggacataagctcgaataatttctgtagtttggtggatgcttccgttatgttatacgacgaagaacaccgtgatcttcttccaaagaaagtaaactccagatgccaagcgaagacaaagaaacaagaaaaagagagtgacaagagattcttccatctcttccctaaaaaaagaagaacttcggttgttactgtaagaaccctgaacaaaaccaacaaaatctttataggatttctatttcttcatcgctcctcgaccttaacacgatccccggtgattcttctgacccacgtaacgaacttcaagggctttattattcttcttcttcatcgtttcttggagattacaagatcactgctgattacaagatgaaaaacccacaaacccctccttcttcttcaactttacttgttgagtacaacaccgagatgacaaaccctaattctcagacaaacagcaagagaaagtgtagtgctaatggtagtgaatccagtccagagccgtctcttaatgcctttcaacaccctaattcgtaacgactttttgacgcctgttgagtcgagaatcctactagaagaagatgacacggagggagttggagcgactctagtggatccatgggaggtaaagtggggtgtttttttgaagaaacggaagatgaagaaagattcagggaaaggaagcttgaattacgctattatctgcggatggaacgatatcgttgaggctaacgtattggaaaaagtcgatgacatcagtatttggtcttgatcaagtgtggatgatcaaggaagtaagatcttgatcaaggaagtaagatcttgatcaattcacagataatcggacggtcaccatttcacaggtatgcttcttagaattctcctataaacgagagttgaaagatcatgtttagcaactgatacaaaattgacatcaattataatatgggcaattgacaaaaataacaccaaaaaaattttcttccaatattaacacaatatttctaaaatttcacaaatagcactataaacttcaattaaacttaactaaaatataagatagaaaataaaaatgagagcagacactaatctaatattaaattgaatcaaataattttaaatagtggaaatatatactctaaattgaaaataaagaactcaaatataatactacaaactaatatttaaataaaaaatctcttttaaatggtgaaaatatataccctaaattagaaataggaaacccaaatataaaaccataaactaatattaaaatgaatcaaaattttcttaaatgattgaaatatataccctaaattggatatagagaaaccaaatataatattttaaatttatttctaatttattttggttgaatagattttatataaaaattatggtattattgagaaataaattagtgataatatttttagaataaaagctttatagtaagccaagtagtgctatttttgaaagtttatgaagtgtgcgttattattgttcataaaatttattttagtgctatttttaagaatttctcttataataatcgatttaagtattttgattattaacatcagtcagtaataatcttatgcaaattatatgcatcaccaatatataaatcacttatttaagtgatgcaaaatttttttacattatttttttaactaatgcaaatatttataataaatgatgtaaaacaatttttttttttagtgaaagtgaaagtgaattgaaagtgaagactagaaaaatcttgatcgttgcacaagccgagccgatgacagtcccttacacatgtgtttttggagccgatgactgtcatttttagataagtgtggaaagtggcttctatgtttttggagccgatgactgtcatttttagataagtgtggaaagtggcttctattcaaggaaggaaagtcaagccgagtatgatcttgatcaattcacagataatcggacggtcaccatttcacaggtatgcttcttagatttcttcttagatgtcacaggtgacgatgacagtcccttacacatgtgtttttggagctcctcacaggtgacgatgacagtcccttacacaggtgacgatgacagtccctttctttttatttatctttttttttattattgtaagggcctcttagatttctcctataaaacgagagttagaaagatcaagaaaatggtgcacaaaaaaaaaaacgaaagatcaagaaaacaaaagagaataaaactgcaaagatacaaattatggagactgtctccgccgttaaccaaacactccccatctccggcggtgaaccggtgaagttcacgacatactctgccgcagttcacaaagtccttgtcatggtcaacgccggaattctagggttactccagcttgtcagccaacagtcctctgttttggaaacacataaagccgcattcctttgtttctgtgttttcattctattctacgccgttcttcgcgtccgtgaagccatggacgtgcgcctacaacccggtttagtcccaaggctcattggacatgggagccatctcttcggtggtctagccgctcttgtgctcgtctccgtcgttagtaccgcgttttctattgtcctttttcttctctggtttatctggctctccgccgtcgtctacttagagacaaacaaacccagtgcctgtccacctcagttaccaccagtttaacatcttgtggttttatgaatggatgagtgtgagatgaaggttaatgtagaataacaccggtgacgttataaacagaccaatccggtagagactgtatcttggtaactagatgatgtggaagaatattccaaatatatttagttgacgtgaaagaaacagcctataaatcgagaatgctttctatatatgacgatttaatatatatattttccttgtgtgtttttcatactactacatttctgcagtatgctttggttatattattttattggtgaaatttacgtttattgaactgattggttcatacaaaaaccaatgttggcttctaatgggatttgtgatgactaaatgattcagttcacttttctttattgtttaacaaagatgcggataatcaaaagaaaattaaacttaaaaaaaacgctttggtcggtttagtcgttaaactatatataaactctgcaagtgttcatctaaaacattcaaaaatataatgtaacaaaggtttaaaacacgtagttttctttcgttttacaggataattatctgtgcaaaatcacttaaatagagtaaaagataactataatttacctatgcaaaatgagaagtgtttgtagatctaatcaaaagggtatgaatcaaagaaaaagaaaaaaagatttatttatgaatgttcctcgcagtttcccagatcccaacacctagtttcc

SEQ ID NO: 4 - DNA, 1356 bp, synthetic construct
  source 1..1356: mol_type = other DNA
acatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattcgaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtggtggagtaaccctagaattcgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatattaatccaaactactgcagcctgacagacaaatgaggatgcaaacaattttaaagtttatctaacgctagctgttttgtttcttctctctggtgcaccaacgacggcgttttctcaatcataaagaggcttgttttacttaaggccaataatgttgatggatcgaaagaagagggcttttaataaacgagcccgtttaagctgtaaacgatgtcaaaaacatcccacatcgttcagttgaaaatagaagctctgtttatatattggtagagtcgactaagagattggtttagtcccaaggctcattagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatggttattctagaaagcttgtcgacaagggtgggcgcgccgacccagctttcttgtacaaagttggcattataagaaagcattgcttatcaattt

SEQ ID NO: 5 - DNA, 7288 bp, synthetic construct
  source 1..7288: mol_type = other DNA
cctaatccgatcgagtacaaaagtaatgtaggacttagtgactacattagttagttaaaactataatatcgactggacaataattatatgtttgttgctcctttggtcgagacagaaccagaagaaattgagatgtatgtagtgaaagagttggtgatagaactaatagtgattaatttgttcagagacagagttgtgtaaactatgaagataatgttggtggactgagaatgtataaatttaacagtggctatgtggttattcttttgattttctttaaaatttcatattaattgaagaaacaatatgtattttcaagaaaatcaaaagacacagacaacaaagttagtgattgagatggaaatgtcaaaccgtttaaaaataagaaaccacaaaataattaaagttttttctatttttgtcaactatagaatgaaaattttggtatttttatcgacataatctagagcatggattggtttttttttttctgggatgaactgttatttttattattcagaccaaatctgagaattatttacaaactagtaaccataatgaaaggcaaaaccatagtacatcccagcaagagatgccctagttagcagactccatgcttgtgaaacaaggatggccataagctcggagtcggaccgaggcaacctgtttagaatcgttttattatttctatcaaacgggcaaagtgacttgtcgagtaaaagattttcttttcaatgctctcatttctatgccaaacattctctctttcagttgtatttgtctaactcgaaaatcttcaggtatatattaagagaatttagcaaaagttaattggatagtatatataactgaatgtagcttagctttttagcgcagaaaaaggcaattttcatgagaatagaattcctacaataatgttcaatgaaacaaatggtcatgtgcccaccaagtccccagcttcagcttcatatcaatcaactataacttggcatatgtgagaaagctttgtggatgtaagaagctccatgattatatcggaagctttgtcgggtataggacaatctttgaaacttagagtgagtttttttagtactgctgaattctcaagaaagtaattcgctagattaatcccagtttcctctcttatattcaggtttttaatcttaacatattccagagtcgatgttaaacatcgaggcacacttgtaaggtcgatttgccctggcaccggttcctgcgaaaatatacaaacaaactgagtttacaatatatttcaacaatgttcttaggggtaacgggtatgcacttttagtatataggctgtaaacttttacttcaaaaaagttatgtaaagaattttactctaacataatagactgtcaagattaggcttttcagatttgggcagctctcaagaaaggctgtcaacaattgtaacgatgtactgaagaaagaagcctccaaatgtgacaagttatggaatgtgggaatctgcagtaccattttcgagtaatggtaaaggacctgcaaagtagaaagaaaatgttttcatggaaagttaaattgttacatatgcatcaaagagtatatgtatatatctatacctctagagtatcattagagatgatcatatgtcttacaccagaaataccggtgagaaagtcacggattatatctctcttacttaaatccaccggttccaaaggactagtgtcaacattgacattaaagtcagtgtcgaggtctattttgaccaaggaactcaaattctttaccacgatcccagcagacagatcatctctaaaactcatatacttgagtctcggggcatcgatctcaactgaaaaaactgtactatacattccccaatagaacgttaaacaaaagatcttcagagtttgagacctcacacgcaaaacagagtgagattcttcattatcaaaattaggaactctcatcatagtaaattcttcaaggacagggcatcctaagaggagcttctctgtaatcttcgacggaccatcgtcgtaataaacgttgttgagatgcatgatcttgagacaaggtaaagaaacaacaagctcttgatgcttcattatcccaacatttgcaagctttaaagaaaccaatgtcttgctcttataaatattcttaggcatgaattctttaataaagataagattcgtgctcccaacatctagatgttgaactccgcgatcaaccgcagtggcgatccactccaagaatctgttggtatatacagtgttaccgccaaagtttaccttgaagtattgcatgtataacccacgattaaactccatatatttgttcacaaccatctgggaggctattccattaggagggaagttacagttctttaagtccaatccgacaacctttagccagacaaactcccatctcttagataaaacgcttgtcttaatcgaatctttgatcccaaggtatgagactatcttagctaccaaatgatctggtaatgcgctgattctatcgcactccattatcgctacaaaaacaaggacaatatcaacaagaagaacgaaccatatgccttgagagtttctcaattaattccaaaaactagggtttattaaagtcggttaaaaaacaacatgagctagttctatagcactctatcgcttaaagataaagaaaaacaacgacaaaataaaccaacaaagtcgaatcatatgccttgagagtttgttgagtatacttacaatcgagggaggaggaatgaagaacgtaactaaccgacggtcaagtgaatatggccggagatctagctttaaggtgagcaagtgatacctactatatagtttatatagtttttcataaagacctttacaaggataacatttacaaaaacatttaccattataattaaatacattagctttttaatatatttcttaattttattaattatacatagatatgttttctattttattgagaacaaaatcttctcaaaatctagttgtcaaattttcattcaacttttaattataaaaaataagaaatttaaaagtaacttgtattcaaaaatgtcaaattaacaaaaaaattgtggtctcacacattattttctaactgctattcttaaattcgcaactaatatttgcttaatcaaatttactttcattttgaaataattatattttatccaaaaaaaggactattgtaaaataaatcaagattttttaaactaatattataatatatcaagatatttttatataattccattaaaaatctataatccccatcaaaattgattctatcatttgattattatttattaagattatatatcttattatataaatcttagttttcaaatatgtaaggaatataatttaacatatattaagtactcaataaaatttagttgcaaatatttatgtgtttatgatatcaacatattcaactaaacaattaataaggaggtaacatgtgttttaaaaattgtatgaaatgtgaaagtttgctcactcatattttgatcatccagttccagctaaactaaagaagttaaggtcccataaccgagaagtttgtcaaaagtttttattagatatttttaaatttaagaaacagataaactttacatggatttatcaatatcttcagatactttagataagtccattgaccacggcaaaggattattttaaagtacgtgaaaaaacattaagaaacagaaacaaaaagaaaaatgaaaagtttcgggaatgtttggatatcaagtaaataaaatgaattcgaagccttctctctttagaacttcgagtttttcgtctgataacgatgtttacgtattattatgatcatctcgcggctgcggagaaggacataagctcgaataatttctgtagtttggtggatgcttccgttatgttatacgacgaagaacaccgtgatcttcttccaaagaaagtaaactccagatgccaagcgaagacaaagaaacaagaaaaagagagtgacaagagattcttccatctcttccctaaaaaaagaagaacttcggttgttactgtaagaaccctgaacaaaaccaacaaaatctttataggatttctatttcttcatcgctcctcgaccttaacacgatccccggtgattcttctgacccacgtaacgaacttcaagggctttattattcttcttcttcatcgtttcttggagattacaagatcactgctgattacaagatgaaaaacccacaaacccctccttcttcttcaactttacttgttgagtacaacaccgagatgacaaaccctaattctcagacaaacagcaagagaaagtgtagtgctaatggtagtgaatccagtccagagccgtctcttaatgcctttcaacaccctaattcgtaacgactttttgacgcctgttgagtcgagaatcctactagaagaagatgacacggagggagttggagcgactctagtggatccatgggaggtaaagtggggtgtttttttgaagaaacggaagatgaagaaagattcagggaaaggaagcttgaattacgctattatctgcggatggaacgatatcgttgaggctaacgtattggaaaaagtcgatgacatcagtatttggtcttgatcaagtgtggatgatcaaggaagtaagatcttgatcaaggaagtaagatcttgatcaattcacagataatcggacggtcaccatttcacaggtatgcttcttagaattctcctataaacgagagttgaaagatcatgtttagcaactgatacaaaattgacatcaattataatatgggcaattgacaaaaataacaccaaaaaaattttcttccaatattaacacaatatttctaaaatttcacaaatagcactataaacttcaattaaacttaactaaaatataagatagaaaataaaaatgagagcagacactaatctaatattaaattgaatcaaataattttaaatagtggaaatatatactctaaattgaaaataaagaactcaaatataatactacaaactaatatttaaataaaaaatctcttttaaatggtgaaaatatataccctaaattagaaataggaaacccaaatataaaaccataaactaatattaaaatgaatcaaaattttcttaaatgattgaaatatataccctaaattggatatagagaaaccaaatataatattttaaatttatttctaatttattttggttgaatagattttatataaaaattatggtattattgagaaataaattagtgataatatttttagaataaaagctttatagtaagccaagtagtgctatttttgaaagtttatgaagtgtgcgttattattgttcataaaatttattttagtgctatttttaagaatttctcttataataatcgatttaagtattttgattattaacatcagtcagtaataatcttatgcaaattatatgcatcaccaatatataaatcacttatttaagtgatgcaaaatttttttacattatttttttaactaatgcaaatatttataataaatgatgtaaaacaatttttttttttagtgaaagtgaaagtgaattgaaagtgaagactagaaaaatcttgatcgttgcacaagccgagccgatgacagtcccttacacatgtgtttttggagccgatgactgtcatttttagataagtgtggaaagtggcttctatgtttttggagccgatgactgtcatttttagataagtgtggaaagtggcttctattcaaggaaggaaagtcaagccgagtatgatcttgatcaattcacagataatcggacggtcaccatttcacaggtatgcttcttagatttcttcttagatgtcacaggtgacgatgacagtcccttacacatgtgtttttggagctcctcacaggtgacgatgacagtcccttacacaggtgacgatgacagtccctttctttttatttatctttttttttattattgtaagggcctcttagatttctcctataaaacgagagttagaaagatcaagaaaatggtgcacaaaaaaaaaaacgaaagatcaagaaaacaaaagagaataaaactgcaaagatacaaattatggagactgtctccgccgttaaccaaacactccccatctccggcggtgaaccggtgaagttcacgacatactctgccgcagttcacaaagtccttgtcatggtcaacgccggaattctagggttactccagcttgtcagccaacagtcctctgttttggaaacacataaagccgcattcctttgtttctgtgttttcattctattctacgccgttcttcgcgtccgtgaagccatggacgtgcgcctacaacccggtttagtcccaaggctcattggacatgggagccatctcttcggtggtctagccgctcttgtgctcgtctccgtcgttagtaccgcgttttctattgtcctttttcttctctggtttatctggctctccgccgtcgtctacttagagacaaacaaacccagtgcctgtccacctcagttaccaccagtttaacatcttgtggttttatgaatggatgagtgtgagatgaaggttaatgtagaataacaccggtgacgttataaacagaccaatccggtagagactgtatcttggtaactagatgatgtggaagaatattccaaatatatttagttgacgtgaaagaaacagcctataaatcgagaatgctttctatatatgacgatttaatatatatattttccttgtgtgtttttcatactactacatttctgcagtatgctttggttatattattttattggtgaaatttacgtttattgaactgattggttcatacaaaaaccaatgttggcttctaatgggatttgtgatgactaaatgattcagttcacttttctttattgtttaacaaagatgcggataatcaaaagaaaattaaacttaaaaaaaacgctttggtcggtttagtcgttaaactatatataaactctgcaagtgttcatctaaaacattcaaaaatataatgtaacaaaggtttaaaacacgtagttttctttcgttttacaggataattatctgtgcaaaatcacttaaatagagtaaaagataactataatttacctatgcaaaatgagaagtgtttgtagatctaatcaaaagggtatgaatcaaagaaaaagaaaaaaagatttatttatgaatgttcctcgcagtttcccagatcccaacacctagtttcc

SEQ ID NO: 6 - DNA, 22 bp, synthetic construct
  source 1..22: mol_type = other DNA
tggagtaaccctagaattccgg

SEQ ID NO: 7 - DNA, 22 bp, synthetic construct
  source 1..22: mol_type = other DNA
gtttagtcccaaggctcattgg

SEQ ID NO: 8 - DNA, 39 bp, synthetic construct
  source 1..39: mol_type = other DNA
atatatggtctcgattgtggagtaaccctagaattcgtt

SEQ ID NO: 9 - DNA, 41 bp, synthetic construct
  source 1..41: mol_type = other DNA
tgtggagtaaccctagaattcgttttagagctagaaatagc

SEQ ID NO: 10 - DNA, 43 bp, synthetic construct
  source 1..43: mol_type = other DNA
aacatgagccttgggactaaaccaatctcttagtcgactctac

SEQ ID NO: 11 - DNA, 37 bp, synthetic construct
  source 1..37: mol_type = other DNA
attattggtctcgaaacatgagccttgggactaaacc

SEQ ID NO: 12 - DNA, 46 bp, synthetic construct
  source 1..46: mol_type = other DNA
cgaattccgaattcgagctcgatctggtaatgcgctgattctatcg

SEQ ID NO: 13 - DNA, 41 bp, synthetic construct
  source 1..41: mol_type = other DNA
ttcatttcgaaggtaccggaaactaggtgttgggatctggg

SEQ ID NO: 14 - DNA, 20 bp, synthetic construct
  source 1..20: mol_type = other DNA
atggagactgtctccgccgt

SEQ ID NO: 15 - DNA, 19 bp, synthetic construct
  source 1..19: mol_type = other DNA
ttaaactggtggtaactga

SEQ ID NO: 16 - DNA, 19 bp, synthetic construct
  source 1..19: mol_type = other DNA
atggagactgtctccgccg

SEQ ID NO: 17 - DNA, 21 bp, synthetic construct
  source 1..21: mol_type = other DNA
gtaggattctggtgtgtgcgc

SEQ ID NO: 18 - DNA, 22 bp, synthetic construct
  source 1..22: mol_type = other DNA
aactctcccgctatgtatgtcg

SEQ ID NO: 19 - DNA, 20 bp, synthetic construct
  source 1..20: mol_type = other DNA
aaccctcgtagattggcaca

SEQ ID NO: 20 - DNA, 18 bp, synthetic construct
  source 1..18: mol_type = other DNA
caccgactacctgatgaa

SEQ ID NO: 21 - DNA, 17 bp, synthetic construct
  source 1..17: mol_type = other DNA
cagcttctccttgatgt

SEQ ID NO: 22 - DNA, 40 bp, synthetic construct
  source 1..40: mol_type = other DNA
cgaattccgaattcgagctccctaatccgatcgagtacaa

(19) United States
(12) Patent Application Publication
(10) Pub. No.: US 2026/0167984 A1
(43) Pub. Date: Jun. 18, 2026

(54) DISEASE RESISTANCE-RELATED PROTEIN RCR1, CODING GENE THEREOF, AND USE THEREOF

(71) Applicant: NSTITUTE OF GENETICS AND DEVELOPMENTAL BIOLOGY, CHINESE ACADEMY OF SCIENCES, Beijing (CN)

(72) Inventors: Jianmin ZHOU, Beijing (CN); Wei Wang, Beijing (CN); Wenjing ZHANG, Beijing (CN); Yun HUANG, Beijing (CN); Chao ZHANG, Beijing (CN)

(73) Assignee: NSTITUTE OF GENETICS AND DEVELOPMENTAL BIOLOGY, CHINESE ACADEMY OF SCIENCES, Beijing (CN)

(21) Appl. No.: 19/126,666

(22) PCT Filed: Oct. 27, 2023
(86) PCT No.: PCT/CN2023/127263

§ 371 Date: May 2, 2025

(30) Foreign Application Priority Data
Nov. 2, 2022 (JP) 202211360611.8

(51) Int. Cl. C12N 15/82

(52) U.S. Cl. CPC C12N 15/8282; C12N 15/8245; C12N 15/8247

(57) ABSTRACT

A disease resistance-related protein RCR1, a coding gene thereof, and use thereof are provided. The disease resistance-related protein RCR1 provided is a protein set forth in SEQ ID NO: 2. When RCR1-transgenic Arabidopsis thaliana or Brassica napus is infected with Plasmodiophora brassicae, the resistance to Plasmodiophora brassicae is significantly enhanced as compared with the wild-type Arabidopsis thaliana or Brassica napus; and the resistance to Plasmodiophora brassicae of RCR1 gene-knock out Arabidopsis thaliana is significantly reduced compared with the wild-type Arabidopsis thaliana. It is indicated that the RCR1 gene is related to the resistance of Arabidopsis thaliana to Plasmodiophora brassicae, and the RCR1 protein and the coding gene thereof can be used for cultivating clubroot-resistant varieties in agricultural production.

### CROSS REFERENCE TO THE RELATED APPLICATIONS

This application is the national phase entry of International Application No. PCT/CN2023/127263, filed on Oct. 27, 2023, which is based upon and claims priority to Chinese Patent Application No. 202211360611.8, filed on Nov. 2, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named KJ0108S_SequenceListing.xml, created on 04/30/2025, and is 34,421 bytes in size.

### TECHNICAL FIELD

The present invention belongs to the field of plant genetic engineering, and specifically relates to a disease resistance-related protein resistance to clubroot 1 (RCR1), an encoding gene, and use thereof.

### BACKGROUND

Brassicaceae plants, including Brassica napus(B. napus), are crucial sources of oilseeds, In recent years, clubroot, a disease caused by Plasmodiophora brassicae(P. brassicae) in cruciferous oilseed crops, has significantly impacted the yield and quality of these crops.

The model plant Arabidopsis thaliana(A. thaliana) also belongs to the Brassicaceae family. As research into the interaction mechanism between A. thaliana and P. brassicae continues to deepen, utilizing disease-resistant genes for breeding resistant varieties has emerged as the most effective, safe, and economical strategy for experts to manage clubroot in crops such as B. napus.

### SUMMARY

The technical problem to be solved by the present invention is how to regulate plant disease resistance and cultivate a disease-resistant plant variety.

In a first aspect, the present invention provides a DNA molecule.

The DNA molecule provided by the present invention includes, in order from the 5′ end, a transcription regulatory element of RCR1 gene and a RCR1 gene;
wherein the transcription regulatory element of RCR1 gene is as follows A1) or A2):
A1) a DNA molecule as shown at positions 1-3643 of SEQ ID NO: 3;
A2) a DNA molecule having 50% or higher than 50% identity to the nucleotide sequence defined in A1) and having transcriptional regulatory function;
and the RCR1 gene is as follows B1) or B2):
B1) a DNA molecule as shown at positions 3644-4090 of SEQ ID NO: 3;
B2) a DNA molecule having 50% or higher than 50% identity to the nucleotide sequence defined in B1) and having the same function as the nucleotide sequence defined in B1).

Further, the DNA molecule includes, in order from the 5′ end, the transcription regulatory element of RCR1 gene, the RCR1 gene, and a 3′UTR region

The nucleotide sequence of the 3′UTR region is as follows C1) or C2):
C1) a DNA molecule as shown at positions 4091-4800 of SEQ ID NO: 3;
C2) a DNA molecule having 50% or higher than 50% identity to the nucleotide sequence defined in C1) and having the same function as the nucleotide sequence defined in C1).

Furthermore, the DNA molecule is composed of the transcription regulatory element of RCR1 gene, the RCR1 gene, and the 3′UTR region in order from the 5′ end.

The nucleotide sequence of the DNA molecule is as follows D1) or D2):
D1) a DNA molecule of SEQ ID NO: 3;
D2) a DNA molecule having 50% or higher than 50% identity to the nucleotide sequence defined in D1) and having the same function as the nucleotide sequence defined in D1).

In A2) or B2) or C2) or D2) mentioned above, the identity includes a nucleotide sequence that has 50% or higher, or 55% or higher, or 60% or higher, or 65% or higher, or 70% or higher, or 75% or higher, or 80% or higher, or 85% or higher, or 90% or higher, or 95% or higher identity with the nucleotide sequence shown at positions 1-3643 of SEQ ID NO: 3 of the present invention, or with the nucleotide sequence shown at positions 3644-4090 of SEQ ID NO: 3 of the present invention, or with the nucleotide sequence shown at positions 4091-4800 of SEQ ID NO: 3 of the present invention, or with the nucleotide sequence of SEQ ID NO: 3 of the present invention. Identity can be evaluated with the naked eye or computer software. Using computer software, the identity between two or more sequences can be expressed as a percentage (%), which can be used to evaluate the identity between related sequences.

In a second aspect, the present invention provides a biological material related to the aforementioned DNA molecule.

The biological material related to the aforementioned DNA molecule provided by the present invention is a recombinant vector containing the above-mentioned DNA molecule, a recombinant microorganism containing the above-mentioned DNA molecule, or a transgenic plant cell line containing the above-mentioned DNA molecule.

The vector may be a plasmid, a cosmid, a phage, or a viral vector. The recombinant vector may be a vector containing the above-mentioned DNA molecule.

The microorganism may be yeast, bacteria, algae or fungi, such as Agrobacterium tumefaciens(A. tumefaciens). The recombinant microorganism may be a microorganism containing the aforementioned DNA molecule or the aforementioned recombinant vector.

In a third aspect, the present invention provides a new use of the aforementioned DNA molecule or biological material related to the aforementioned DNA molecule.

The present invention provides a use of the aforementioned DNA molecule or the biological material related to the aforementioned DNA molecule in the following M1) or M2):
M1) regulating plant disease resistance;
M2) cultivating a transgenic plant or gene-edited plant with enhanced disease resistance.

In the above use, the regulating plant disease resistance is to improve plant disease resistance; the enhanced disease resistance is embodied in: a susceptible plant containing the aforementioned DNA molecule become disease-resistant; further embodied in: a plant susceptible to clubroot containing the above DNA molecule shows a clubroot resistance.

In a fourth aspect, the present invention provides a new use of the plant disease resistance-related protein RCR1.

The present invention provides use of RCR1 protein in following M1) or M2):
M1) regulating plant disease resistance;
M2) cultivating a transgenic plant or gene-edited plant with enhanced disease resistance; and the RCR1 protein is N1) or N2) or N3) or N4):
N1) a protein having an amino acid sequence of SEQ ID NO: 2;
N2) a fusion protein obtained by attaching a tag to the N-terminus and/or C-terminus of the protein of SEQ ID NO: 2;
N3) a plant disease resistance related protein obtained from the amino acid sequence of SEQ ID NO: 2 by substitution, and/or deletion, and/or addition of one or more amino acid residues;
N4) a plant disease resistance related protein having 75% identity to the amino acid of SEQ ID NO: 2.

Among them, SEQ ID NO: 2 consists of 148 amino acid residues.

Among the protein described in N2) above, the tag refers to a polypeptide or protein that is fused and expressed together with the target protein using DNA in vitro recombination technology to facilitate the expression, detection, tracing and/or purification of the target protein. The tag may be a Flag tag, His tag, MBP tag, HA tag, myc tag, GST tag and/or SUMO tag, etc.

In the protein described in N3) above, the substitution and/or deletion and/or addition of one or more amino acid residues is a substitution and/or deletion and/or addition of no more than 35 amino acid residues, or no more than 30 substitution and/or deletion and/or addition of amino acid residues, or no more than 25 substitutions and/or deletions and/or additions of amino acid residues, or no more than 20 substitutions and/or deletions and/or additions of amino acid residues, or no more than 15 substitutions and/or deletions and/or additions of amino acid residues, or no more than 10 substitutions and/or deletions and/or additions of amino acid residues, or no more than 9 substitutions and/or deletions and/or additions of amino acid residues, or no more than 8 substitutions and/or deletions and/or additions of amino acid residues, or no more than 7 substitutions and/or deletions and/or additions of amino acid residues, or no more than 6 substitutions and/or deletions and/or additions of amino acid residues, or no more than 5 substitutions and/or deletions and/or additions of amino acid residues, or no more than 4 substitutions and/or deletions and/or additions of amino acid residues, or no more than 3 substitutions and/or deletions and/or additions of amino acid residues, or no more than 2 substitutions and/or deletions and/or additions of amino acid residues, or no more than 1 substitutions and/or deletions and/or additions of amino acid residues.

In the protein described in N4) above, “identity” includes a amino acid sequence having 75% or higher, or 80% or higher, or 85% or higher, or 90% or higher, or 91% or higher, or 92% or higher, or 93% or higher, or 94% or higher, or 95% or higher, or 96% or higher, or 97% or higher, or 98% or higher, or 99% or higher identity to the amino acid sequence of SEQ ID NO: 2.

The protein described in the above N1) or N2) or N3) or N4) can be artificially synthesized, or obtained by firstly encoding its coding gene can be synthesized first and then biologically expressed.

In a fifth aspect, the present invention provides a new use of a biological material related to RCR1 protein.

The present invention provides the use of the biological material related to RCR1 protein in the following M1) or M2):
M1) regulating plant disease resistance;
M2) cultivating a transgenic plant or gene-edited plant with enhanced disease resistance;
the biological material related to RCR1 protein is a nucleic acid molecule encoding the RCR1 protein, or an expression cassette including the nucleic acid molecule, a recombinant vector including the nucleic acid molecule, a recombinant microorganism including the nucleic acid molecule, or a transgenic plant cell line including the nucleic acid molecule.

In the above use, the nucleic acid molecule encoding the RCR1 protein is a DNA molecule represented by the following P1) or P2) or P3):
P1) a DNA molecule shown in SEQ ID NO: 1;
P2) a DNA molecule shown at positions 3644-4090 of SEQ ID NO: 3;
P3) a DNA molecule that has 50% or more identity to the nucleotide sequence defined by P1) or P2) and encodes the RCR1 protein.

Among them, the nucleic acid molecule can be DNA, such as cDNA, genomic DNA or recombinant DNA; the nucleic acid molecule can also be RNA, such as mRNA or hnRNA, etc.

Those of ordinary skill in the art can easily mutate the nucleotide sequence encoding the RCR1 protein of the present invention using known methods, such as directed evolution and point mutation. Those artificially modified nucleotides that have 50% or higher identity with the nucleotide sequence encoding the RCR1 protein, as long as they encode the RCR1 protein and have the same function, are derived from the nucleotide sequence of the present invention and are equivalent to Sequences of the invention.

In the above P3), the identity includes a nucleotide sequence having 50% or higher, or 55% or higher, or 60% or higher, or 65% or higher, or having 70% or higher, or having 75% or higher, or having 80% or higher, or having 85% or higher, or having 90% or higher, or 95% or higher identity to the nucleotide sequence of the protein consisting of the amino acid sequence shown in the coding SEQ ID NO: 2 of the present invention. Identity can be assessed with the naked eye or with computer software. Using computer software, the identity between two or more sequences can be expressed as a percentage (%), which can be used to evaluate the identity between related sequences.

The expression cassette refers to DNA capable of expressing RCR1 protein in a host cell. The DNA may not only include a promoter that initiates RCR1 transcription, but may also include a terminator that terminates RCR1 transcription. Furthermore, the expression cassette may also include an enhancer sequence. Promoters useful in the present invention include, but are not limited to: constitutive promoters; tissue, organ and development specific promoters and inducible promoters. Suitable transcription terminators include, but are not limited to: terminator of A. tumefaciens nopaline synthase (NOS terminator), cauliflower mosaic virus CaMV 35S terminator, tml terminator, pea rbcS E9 terminator and nopaline and octopine synthase terminator. In a specific embodiment of the present invention, the expression cassette can be the DNA molecule shown in SEQ ID NO: 3. In another specific embodiment of the present invention, the expression cassette can be the DNA molecule shown in SEQ ID NO: 5.

The vector may be a plasmid, cosmid, phage or viral vector. The recombinant vector may be a vector containing the RCR1 gene expression cassette constructed using an existing plant expression vector. The plant expression vectors include binary A. tumefaciens vectors and vectors that can be used for plant gene gun transformation, etc, such as pAHC25, pBin438, pCAMBIA1302, pCAMBIA2301, pCAMBIA1301, pCAMBIA1300, pBI121, pCAMBIA1391-Xa or pCAMBIA1391-Xb, etc. The plant expression vector may also contain the 3′ untranslated region of the foreign gene, that is, containing the poly(A) signal and any other DNA fragments involved in mRNA processing or gene expression. The poly(A) signal can guide poly(A) to be added to the 3′ end of the mRNA precursor. For example, the untranslated regions transcribed at the 3′ end of storage protein genes of the A. tumefaciens crown gall tumor induction (Ti) plasmid gene (such as nopaline synthase gene Nos), plant genes (such as soybean) all have similar functions. When using the gene of the present invention to construct a plant expression vector, enhancers including translation enhancers or transcription enhancers can also be used. These enhancer regions can be ATG start codons or adjacent region start codons, but must be the same as the reading frame of the coding sequence to ensure correct translation of the entire sequence. The translation control signals and initiation codons come from a wide range of sources, and may be natural or synthetic. The translation initiation region can be derived from the transcription initiation region or from a structural gene. To facilitate the identification and screening of transgenic plant cells or plants, the plant expression vector can be modified. For example, genes that can express in plants and encode enzyme that can produce color changes or luminescent compounds in plants (such as the GUS gene and luciferase gene), antibiotic marker genes (like nptII for kanamycin and related antibiotics resistance, bar for glufosinate herbicide resistance, hph for hygromycin resistance, dhfr for methotrexate resistance, and EPSPS for glyphosate resistance), or chemically resistant marker genes (such as herbicide resistance genes), genes for invertase, which provide the ability to metabolize mannose, can be added. Additionally, the, can be included Considering the safety of transgenic plants, the transformed plants can be directly screened under adverse conditions without adding any selective marker genes.

The microorganism may be yeast, bacteria, algae or fungi, such as A. tumefaciens. The recombinant microorganism may be a microorganism containing the above-mentioned expression cassette or the above-mentioned recombinant vector.

In the above use, the regulation of plant disease resistance is to improve plant disease resistance; the improvement of plant disease resistance is reflected in: the higher the RCR1 protein content and/or activity in the plant or the higher the RCR1 gene expression, the greater the resistance of the plant; further reflected in: the higher the RCR1 protein content and/or activity in the plant or the higher the RCR1 gene expression, the higher the plant's resistance to P. brassicae.

In a sixth aspect, the present invention provides a new use of the substance represented by Q1) or Q2):
Q1) Substance that inhibits or reduces the activity and/or content of RCR1 protein in a plant;
Q2) Substance that inhibits the expression of the RCR1 protein-encoding gene in a plant or knocks out the RCR1 protein-encoding gene in a plant.

The present invention provides use of the substance represented by Q1) or Q2) in reducing plant disease resistance or cultivating a transgenic plant or gene-edited plant with reduced disease resistance.

In the above use, the substance that inhibits or reduces the activity and/or content of RCR1 protein in a plant can be any substance that can cause the loss of RCR1 protein activity in plants, such as proteins, polypeptides or small molecule compounds (such as protein activity inhibitors) that inhibit RCR1 protein synthesis or promote RCR1 protein degradation or inhibit RCR1 protein function.

The substance that inhibits the expression of the RCR1 protein-encoding gene can be any substance that can prevent the expression of the RCR1 protein-encoding gene in plants, such as substances that silence the RCR1 protein-encoding gene in plants (such as miRNA, siRNA, dsRNA, shRNA, etc.).

The substance that knocks out the RCR1 protein-encoding gene can be any material that achieves the functional protein product of the RCR1 gene not being produced by the host cell. Specific methods include removing all or part of the coding gene sequence, introducing frameshift mutations to prevent the production of functional proteins, removing or altering regulatory components (such as promoter editing) so that the coding gene sequence is not transcribed, and preventing translation by binding to mRNA. Typically, knock outs are performed at the level of genomic DNA, so that future generations of cells also permanently carry the knock out.

Further, the substance for knocking out the RCR1 protein-encoding gene can be any substance that can mutate the RCR1 protein-encoding gene in plants (the mutation form can be deletion mutation and/or insertion mutation and/or base substitution), thereby losing activity, such as zinc finger protein ZFN gene editing system or TALENs gene editing system or CRISPR/Cas9 gene editing system, etc.

Furthermore, the material that knocks out the RCR1 protein-encoding gene is a CRISPR/Cas9 gene editing vector that knocks out the RCR1 protein-encoding gene in plants.

In a specific embodiment of the present invention, the CRISPR/Cas9 gene editing vector for knocking out the RCR1 protein-encoding gene in plants is a recombinant vector pHEE401E-RCR1, and the recombinant vector pHEE401E-RCR1 is a vector obtained by inserting the DNA molecule shown in SEQ ID NO: 4 at the BsaI restriction site of the pHEE401E vector.

In the above use, the reduction of plant disease resistance is reflected in: disease-resistant plants with RCR1 protein deletion or RCR1 gene knock out showing disease susceptibility; further reflected in clubroot-resistant plants with RCR1 protein deletion or RCR1 gene knock out showing infecting clubroot disease.

In a seventh aspect, the present invention provides a method for cultivating a transgenic plant or gene-edited plant with improved disease resistance.

The method for cultivating a transgenic plant or gene-edited plant with improved disease resistance provided by the present invention including the steps of increasing the content and/or activity of RCR1 protein in a recipient plant to obtain a transgenic plant or gene-edited plant; the transgenic plant or gene-edited plant; the disease resistance of the transgenic plant or gene-edited plant being higher than that of the recipient plant.

Further, the disease resistance of the transgenic plant or gene-edited plant is higher than that of the recipient plant, as reflected in any one of the following X1)-X2):
X1) an incidence rate of the transgenic plant or gene-edited plant lower than that of the recipient plant;
X2) a disease index of the transgenic plant or gene-edited plant lower than that of the recipient plant.

Furthermore, the method for increasing the content and/or activity of RCR1 protein in a recipient plant is to overexpress RCR1 protein in a recipient plant.

The overexpression method may be to introduce an encoding gene of RCR1 protein into the recipient plant.

Furthermore, the encoding gene of RCR1 protein is introduced into the recipient plant through a recombinant vector.

The recombinant vector includes a promoter for initiating expression of the encoding gene of RCR1 protein.

The encoding gene of RCR1 protein is shown in SEQ ID NO: 3 at positions 3644-4090.

The nucleotide sequence of the promoter is shown in positions 1-3643 of SEQ ID NO: 3, or positions 1-6131 of SEQ ID NO: 5.

In a specific embodiment of the present invention, the encoding gene of RCR1 protein is introduced into the recipient plant through the recombinant vector pCAMBIA1300-p3.6kRCR1. The recombinant vector pCAMBIA1300-p3.6kRCR1 is a vector obtained after inserting the DNA molecule shown in SEQ ID NO: 3 into the KpnI restriction site of the pCAMBIA1300 vector.

In another specific embodiment of the present invention, the RCR1 protein-encoding gene is introduced into the recipient plant through the recombinant vector pCAMBIA1300-p6kRCR1. The recombinant vector pCAMBIA1300-p6kRCR1 is a vector obtained after inserting the DNA molecule shown in SEQ ID NO: 5 into the KpnI restriction site of the pCAMBIA1300 vector.

In an eighth aspect, the present invention provides a method for cultivating a transgenic plant or gene-edited plant with reduced disease resistance.

The method for cultivating a transgenic plant or gene-edited plant with reduced disease resistance provided by the present invention includes the steps of reducing the activity and/or content of RCR1 protein in a recipient plant to obtain a transgenic plant or gene-edited plant; the transgenic plant or gene-edited plant having disease resistance lower than that of the recipient plant.

Further, the disease resistance of the transgenic plant or gene-edited plant is lower than that of the recipient plant, as shown in any one of the following Y1)-Y2):
Y1) an incidence rate of the transgenic plant or gene-edited plants higher than that of the recipient plant;
Y2) a disease index of the transgenic plant or gene-edited plant higher than that of the recipient plant.

Furthermore, the method for reducing the activity and/or content of RCR1 protein in the recipient plant is achieved by introducing the substance that inhibits or reduces the activity and/or content of RCR1 protein in the plant, or the substance that inhibits the expression of the RCR1 protein encoding gene in the plant, or the substance that knocks out the RCR1 protein encoding gene in the plant, into the recipient plant.

Furthermore, the substance that knocks out the RCR1 protein-encoding gene is a CRISPR/Cas9 gene editing vector that knocks out the RCR1 protein-encoding gene in plants.

In a specific embodiment of the present invention, the CRISPR/Cas9 gene editing vector for knocking out the RCR1 protein-encoding gene in plants is a recombinant vector pHEE401E-RCR1, and the recombinant vector pHEE401E-RCR1 is a vector obtained by inserting the DNA molecule shown in SEQ ID NO: 4 at the BsaI restriction site of the pHEE401E vector.

Transgenic plants or gene-edited plants prepared by any of the above methods also belong to protection scope of the present invention.

In any of the above uses or methods, the plant is a monocotyledonous plant or a dicotyledonous plant, and the dicotyledonous plant can be a cruciferous plant; the cruciferous plant can specifically be A. thaliana, B. napus, or cabbage. In specific embodiments of the present invention, the A. thaliana can be Colombian ecotype Col-0 or Est-1; the B. napus can be Zhongshuang 11 (ZS11).

In any of the above-mentioned applications or methods, the disease resistance is resistance to clubroot fungus; the clubroot fungus is specifically the physiological race No. 4 of clubroot fungus.

### BRIEF DESCRIPTION OF THE DRAWINGS

FIGS. 1A-1C show the resistance analysis of transgenic A. thaliana plants to clubroot disease. FIG. 1A shows Est-1, RCR1-knock out plants rcr1-1 and rcr1-2, Col-0, and RCR1 overexpression plants p3.6kRCR1_Col-0 #1 and p3.6kRCR1_Col-0 #2 were inoculated with P. brassicae PbDy, a strain collected from Dayi, Sichuan. The symptoms of the inoculated plants were observed and photographed 21 days post inoculation. Wherein, white line: 1 cm; red line, 1 mm. FIG. 1B and FIG. 1C show incidence rate and disease index (FIG. 1B) and relative biomass (FIG. 1C) (n≥12) of Est-1, RCR1 knock out plants rcr1-1 and rcr1-2, Col-0, and RCR1 overexpression plants p3.6kRCR1_Col-0 #1 and p3.6kRCR1_Col-0 #2 21 days after inoculation with P. brassicae.

FIGS. 2A-2C show the resistance analysis of transgenic B. napus plants to P. brassicae. FIG. 2A shows ZS11 and p3.6kRCR1_ZS11 were inoculated with PbDy, the strain collected from Dayi region, Sichuan and PbXm, the strain collected from Xinmin region, Shenyang. The symptoms of the indicated inoculated plants were observed and photographed 21 days post inoculation. p3.6kRCR1_ZS11 is a transgenic plant expressing p3.6kRCR1 in the Zhongshuang11 (ZS11) background. FIG. 2B shows the incidence rate of ZS11 and p3.6kRCR1_ZS11 21 days after inoculation with P. brassicae (n≥12). FIG. 2C shows ZS11 and p6kRCR1_ZS11 were inoculated with PbDy the strain collected from Dayi region, Sichuan and PbXm, the strain collected from Xinmin region, Shenyang. The symptoms of the indicated inoculated plants were observed and photographed 21 days post inoculation. p6kRCR1_ZS11 #1 and p6kRCR1_ZS11 #2 are transgenic plants expressing p6kRCR1 under the ZS11 background.

### DETAILED DESCRIPTION OF THE EMBODIMENTS

The following examples facilitate a better understanding of the present invention, but do not limit the present invention. The test methods in the following examples are all conventional methods unless otherwise specified. The test materials used in the following examples were all purchased from conventional biochemical reagent stores unless otherwise specified. The quantitative experiments in the following examples were repeated three times, and the results were averaged.

The pCAMBIA1300 vector in the following examples is described in the document “Liang, X X. et al. Ligand-triggered de-repression of Arabidopsis heterotrimeric G proteins coupled to immune receptor kinases. Cell Research 28, 529-543 (2019).” This biological material can be available from the Institute of Genetics and Developmental Biology of the Chinese Academy of Sciences. This biological material is only used to repeat the experiments related to the invention and cannot be used for other purposes.

The pCBC-DT1T2 vector and pHEE401E vector in the following examples are both described in the document “Editor, D. CRISPR-GE: a convenient software toolkit for CRISPR-based genome editing. Mol. Plant 10, 1246-1249 (2017)”. This biological material can be available from the Institute of Genetics and Developmental Biology of the Chinese Academy of Sciences. This biological material is only used to repeat the relevant experiments related to the invention and cannot be used for other purposes.

The A. tumefaciens GV3101 in the following examples is described in the document “Liang, X X. et al. Ligand-triggered de-repression of Arabidopsis heterotrimeric G proteins coupled to immune receptor kinases. Cell Research 28, 529-543 (2019)”. This biological material can be available from the Institute of Genetics and Developmental Biology of the Chinese Academy of Sciences. This biological material is only used to repeat the relevant experiments related to the invention and cannot be used for other purposes.

The B. napus variety Zhongshuang 11 (ZS11) in the following examples is described in the document “Sun, F M. et al. The high-quality genome of Brassica napus cultivar ‘ZS11’ reveals the introgression history in semi-winter morphotype. Plant J 92, 452-468(2017)”. This biological material can be available from the Institute of Genetics and Developmental Biology of the Chinese Academy of Sciences. This biological material is only used to repeat the relevant experiments related to the invention and cannot be used for other purposes.

The P. brassicae PbXm in the following examples is described in the document “Shah, N., Sun, J., Yu, S. et al. Genetic variation analysis of field isolates of clubroot and their responses to Brassica napus lines containing resistant genes CRb and PbBa8.1 and their combination in homozygous and heterozygous state. Mol Breeding 39, 153(2019)”. This biological material can be available from the Institute of Genetics and Developmental Biology, Chinese Academy of Sciences. This biological material is only used to repeat the relevant experiments related to the invention and cannot be used for other purposes.

The amino acid sequence of the RCR1 protein in the following examples is SEQ ID NO: 2, the nucleotide sequence of its encoding gene corresponds to positions 1-447 in SEQ ID NO: 1, and its genome sequence corresponds to positions 3644-4090 in SEQ ID NO: 3.

### Example 1. Obtaining RCR1-Knock Out A. Thaliana and RCR1-Overexpressing A. Thaliana and Analysis of their Resistance to P. brassicae

One. Obtaining RCR1-knock out A. thaliana and RCR1-overexpression A. thaliana

### 1. Construction of RCR1-Knock Out Vector

### 1) Obtaining the Knock Out Vector Target

The website http://skl.scau.edu.cn/ was used to design targets for knocking out the RCR1 gene. The target sequences are as follows:

Target 1:
(SEQ ID NO: 6)
5′-TGGAGTAACCCTAGAATTCCGG-3′;
Target 2:
(SEQ ID NO: 7)
5′-GTTTAGTCCCAAGGCTCATTGG-3′.

### 2) Construction of Vector

Primers were designed according to the designed target. The primer sequences are as follows:

Targte1-BsF:
(SEQ ID NO: 8)
ATATATGGTCTCGATTGTGGAGTAACCCTAGAATTCGTT;
Targte1-F0:
(SEQ ID NO: 9)
TGTGGAGTAACCCTAGAATTCGTTTTAGAGCTAGAAATAGC;
Targte2-R0:
(SEQ ID NO: 10)
AACATGAGCCTTGGGACTAAACCAATCTCTTAGTCGACTCTAC;
Targte2-BsR:
(SEQ ID NO: 11)
ATTATTGGTCTCGAAACATGAGCCTTGGGACTAAACC.

Four-primer PCR amplification was performed using the pCBC-DT1T2 vector as a template, and the target fragment was recovered using agarose gel electrophoresis, and then the recovered target fragment was inserted into the pHEE401E vector using a restriction-ligation cloning approach, generating a recombinant vector pHEE401E-RCR1, that is the RCR1-knock out vector. It was then sequenced.

The system for restriction-ligation cloning was as follows: target fragment 2 μl, pHEE401E vector 2 μl, 10×T4 ligase buffer 1.5 μl, 10×BSA1.5 μl, BsaI1 μl, T4 ligase 11, and sterile water 6 μl.

The sequencing results show that the recombinant vector pHEE401E-RCR1 was a vector obtained by inserting the DNA molecule shown in SEQ ID NO: 4 into the BsaI restriction site of the pHEE401E vector.

### 2. Construction of RCR1 Gene Expression Vector

### 1) Obtaining RCR1 Gene Fragment

The DNA of the ecotype Est-1 of A. thaliana was extracted and amplified by PCR using primers RCR1-F-3.6k and RCR1-R. A PCR product of 4800 bp in size was obtained, and its nucleotide sequence is shown in SEQ ID NO: 3. This sequence, designated as the p3.6kRCR1 gene, encompasses a RCR1 promoter region, a sequence of approximately 3.6 kb, a RCR1 coding region, a sequence of approximately 1.1 kb and a UTR region. The primer sequences are as follows:

RCR1-F-3.6k:
(SEQ ID NO: 12)
5′-CGAATTCCGAATTCGAGCTCGATCTGGTAATGCGCTGATTCTATC
G-3′;
RCR1-R:
(SEQ ID NO: 13)
5′-TTCATTTCGAAGGTACCGGAAACTAGGTGTTGGGATCTGGG-3′.

### 2) Obtaining RCR1 Gene Expression Vector

The pCAMBIA1300 vector was digested with KpnI single enzyme and recovered to obtain the pCAMBIA1300 vector backbone; using in-Funion homologous recombination technology, the pCAMBIA1300 vector backbone was reacted with the PCR product of 4800 bp in size. The expression vector pCAMBIA1300-p3.6kRCR1 was obtained and sequenced.

The sequencing results show that the recombinant vector pCAMBIA1300-p3.6kRCR1 is a vector obtained by inserting the DNA molecule shown in SEQ ID NO: 3 into the KpnI restriction site of the pCAMBIA1300 vector. In the DNA molecule shown in SEQ ID NO: 3, positions 1-3643 correspond to the promoter sequence, positions 3644-4090 correspond to the RCR1 genome sequence, and positions 4091-4800 correspond to the 3′UTR region.

### 3. Obtaining Transgenic A. Thaliana

1) Obtaining A. tumefaciens Carrying pHEE401E-RCR1 or pCAMBIA1300-p3.6kRCR1.

The method of electroporation transformation was used to introduce the recombinant vector pHEE401E-RCR1 obtained in step 1 and the pCAMBIA1300-p3.6kRCR1 obtained in step 2 into A. tumefaciens GV3101. The transformed strain was smeared on an LB solid culture dish containing kanamycin and gentamicin, cultured at 28° C. for 36 hours. Positive monoclonal strains were screened to obtain A. tumefaciens GV3101 carrying pHEE401E-RCR1 and pCAMBIA1300-p3.6kRCR1 of A. tumefaciens GV3101, respectively.

### 2) Obtaining Transgenic A. Thaliana

Using the A. thaliana transformation method mediated by A. tumefaciens, the A. tumefaciens GV3101 carrying pHEE401E-RCR1 obtained in step 1) was transformed into A. thaliana ecotype Est-1 (clubroot-resistant material), and the A. tumefaciens carrying pCAMBIA1300-p3.6kRCR1 obtained in step 1) was transformed into A. thaliana ecotype Col-0 (clubroot-susceptible material). The specific steps were as follows:
I. Preparation of A. thaliana transformation receptors: A. thaliana in the flowering stage were selected, the flowers that have opened were picked, and the remaining A. thaliana buds were used as receptors for A. tumefaciens infection.
II. Cultivation of A. tumefaciens: A small amount of bacterial solution from the A. tumefaciens storage solution was taken and cultured in streaks on LB solid medium (containing 50 mg/l kanamycin and 50 mg/l gentamicin) at 28° C. in the dark for 12 hours. A few bacterial cells were picked and inoculated into 2 ml of LB liquid culture medium (containing the corresponding antibiotics) and cultured at 28° C. for 12 hours. Then 2 ml of LB liquid culture medium was transferred to 300 ml of LB liquid culture medium (containing the corresponding antibiotics) and cultured for about 10 hours, centrifuged at 4,000 rpm to collect the bacterial solution. The bacterial solution was diluted with 5% sucrose solution to an OD of about 0.9, and a surfactant (silwet L-77) was added at a ratio of 17/10000 to prepare for transformation.
III. A. tumefaciens infection of A. thaliana flower buds: The diluted bacterial solution was put into a cylindrical glass bottle. The A. thaliana containing only flower buds was placed upside down in the bacterial solution for 4-5 minutes, and then laid horizontally for 24 hours in the dark. After 24 hours of protection from light, the plant grew vertically.
IV. Screening of resistant plants: The seeds received from the transgenic plants were spread evenly on a ½ MS medium containing 25 mg/L hygromycin B and 50 mg/L carbenicillin for screening, and after 10 days, normal growth seedlings were selected to move to the greenhouse for cultivation.

### 3) Obtaining RCR1-Knock Out A. Thaliana

Transgenic A. thaliana that has grown for about two weeks were selected, and then 1-2 leaves were picked to extract total DNA using the CTAB method, and the DNA fragments in the genome were amplified by using primer RCR1-de-F and primer RCR1-de-R. It was sequenced to determine the mutation site, and finally two different mutant forms of RCR1-knock out A. thaliana lines were obtained, named rcr1-1 and rcr1-2 respectively. The primer sequences are as follows:

RCR1-de-F:
(SEQ ID NO: 14)
5′-ATGGAGACTGTCTCCGCCGT-3′;
RCR1-de-R:
(SEQ ID NO: 15)
5′-TTAAACTGGTGGTAACTGA-3′.

RCR1-knock out A. thaliana rcr1-1 is an A. thaliana mutant with a homozygous mutation in the RCR1 gene (the same mutation occurred in both chromosomes). The only difference in the genome sequence from the wild-type A. thaliana ecotype Est-1 was that a base deletion of 145 bp occurred at positions 116-260 of the gene encoding RCR1 protein (SEQ ID NO: 1).

RCR1-knock out A. thaliana rcr1-2 is an A. thaliana mutant with a homozygous mutation in the RCR1 gene (the same mutation occurred in both chromosomes). The only difference in the genome sequence from the wild-type Arabidopsis ecotype Est-1 was that a base mutation occurred at position 260 of the gene encoding RCR1 protein (SEQ ID NO: 1), and the base T was mutated into base A at this position.

### 4) Obtaining RCR1-Overexpression in A. Thaliana

Transgenic A. thaliana that has grown for about two weeks was selected, and then 1-2 leaves were picked to extract total DNA using the CTAB method, and then PCR was performed to amplify the DNA in the genome to detect the integration of the target gene into the genome. The primer pair used for PCR amplification was RCR1-F and 1300-R. The amplification product was the connection fragment located at the 3′ end of RCR1 and the pCAMBIA1300 vector. The amplified transgenic A. thaliana with a size of approximately 1000 bp was p3.6kRCR1 overexpression A. thaliana, termed as p3.6kRCR1_Col-0. Two independent p3.6kRCR1 overexpression A. thaliana lines were termed p3.6kRCR1_Col-0 #1 and p3.6kRCR1 Col-0 #2, respectively. T₂ generation was used as the experimental materials for the following clubroot resistance analysis. The primer sequences are as follows:

RCR1-F:
(SEQ ID NO: 16)
5′-ATGGAGACTGTCTCCGCCG-3′;
1300-R:
(SEQ ID NO: 17)
5′-GTAGGATTCTGGTGTGTGCGC-3′.

Two. Analysis of resistance of RCR1-knock out A. thaliana and RCR1-overexpression A. thaliana to P. brassicae

1. P. brassicae Inoculation

Est-1, Col-0, and RCR1-knock out A. thaliana rcr1-1, rcr1-2, RCR1 overexpress A. thaliana p3.6kRCR1_Col-0 #1 and p3.6kRCR1_Col-0 #2 obtained in step one, were used as experimental materials for P. brassicae inoculation experiment respectively. Before inoculation, each experimental material was cultured under conventional conditions (12 hours of light, 12 hours of darkness, at a temperature of 22° C.). When the plants had grown for about 14 days, P. brassicae infection experiment was conducted. The specific steps are as follows:
1) The roots pieces of the collected plants containing P. brassicae (recorded as P. brassicae PbDy, identified as physiological race No. 4) from the Dayi region of Sichuan province were cut into small pieces with a blade, added with water, and then broken in a tissue crusher.
2) The mixture containing bacterial solution and broken tissue was filtered by using 8 layers of gauzer to obtain relatively clean P. brassicae PbDy solution.
3) The spore concentration of the P. brassicae PbDy in the bacterial solution was measured by using a hemocytometer.
4) The concentration of P. brassicae PbDy was diluted to 1.0×10⁷/mL, and then the root-drenching method was used to inoculate A. thaliana, that is: 1 mL of the diluted bacterial solution was absorbed by using a pipette, and poured on the roots of A. thaliana.

### 2. Disease Index and Incidence Statistics

The incidence rate and disease index were calculated by using the grading system of A. thaliana P. brassicae. The statistical standards of the grading system of A. thaliana-P. brassicae are as follows: Grade 0, the roots of A. thaliana are not infected and the structure of root system is intact; Grade 1, there are small nodules on the lateral roots and the main root is not affected; Grade 2, there are lumps on the lateral roots and main roots, but some root structures are intact and unaffected; Grade 3, both the main root and lateral roots are infected; Grade 4, the lateral roots and root hairs of A. thaliana are completely damaged, leaving only an enlarged main root in rod shape. The calculation formula of disease incidence=(number of diseased plants/total number of inoculated plants)×100%; the calculation formula of disease index=(Σ(number of disease grade×number of diseased plants at this grade))/(total number of inoculated plants×number of the highest grade 4)×100.

3. Analysis of Relative Biomass of P. brassicae

After inoculation with PbDy for 21 days, the underground parts of each material were collected, the total DNA of the underground parts was extracted using the CTAB method, and then quantitative real-time PCR was used to detect the expression of A. thaliana housekeeping gene At-Actin and P. brassicae housekeeping gene Pb-Actin in different materials. The primer sequences are as follows:

Actin-RT-F:
(SEQ ID NO: 18)
5′-AACTCTCCCGCTATGTATGTCG-3′;
Actin-RT-R:
(SEQ ID NO: 19)
5′-AACCCTCGTAGATTGGCACA-3′;
Pb-Actin-RT-F:
(SEQ ID NO: 20)
5′-CACCGACTACCTGATGAA-3′;
Pb-Actin-RT-R:
(SEQ ID NO: 21)
5′-CAGCTTCTCCTTGATGT-3′.

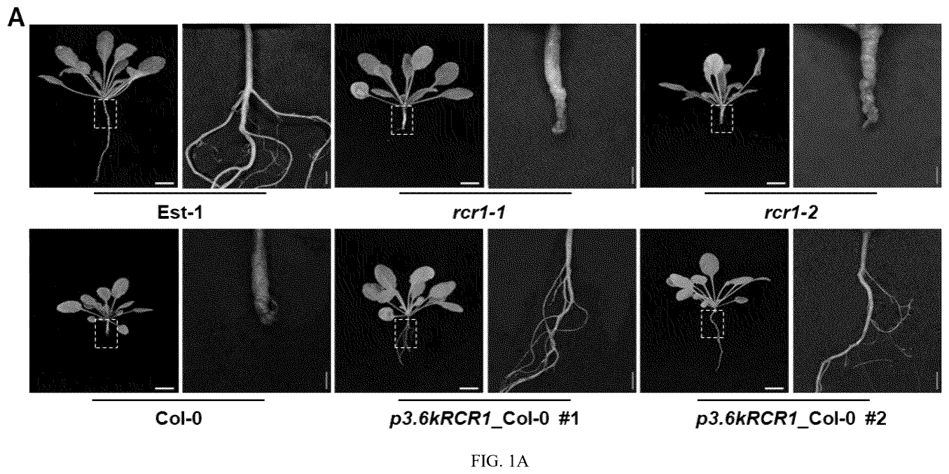

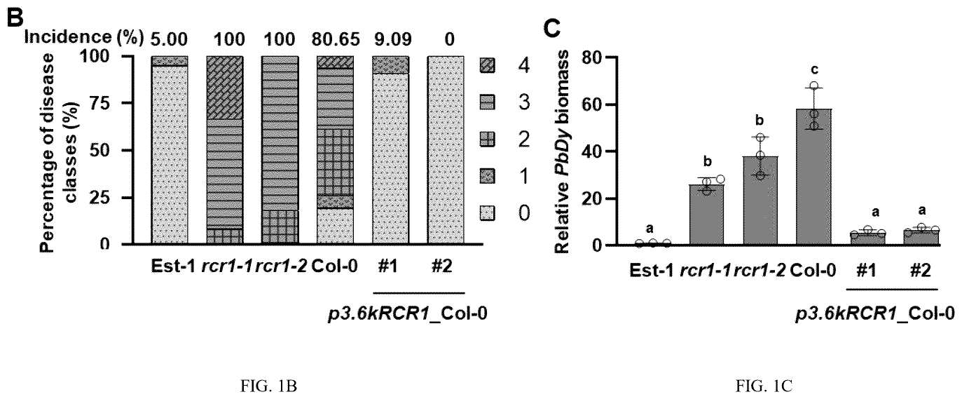

The disease symptoms, incidence rate and disease index of the plants 21 days after the inoculation of the P. brassicae, and the relative biomass of the P. brassicae are shown in FIGS. 1A-1C. The results show that: 21 days after the inoculation, the transgenic material p3.6kRCR1_Col-0 #1 and #2 heterologously expressing RCR1 showed similar phenotypes to the resistant material Est-1, that is, root development was basically normal, and only a small number of plants showed mild symptoms; while in the background of the resistant material Est-1, the two RCR1 gene knock out mutants rcr1-1 and rcr1-2 showed a similar phenotype to the clubroot susceptible material Col-0, that is, spindle-shaped tumors formed in the roots, and almost all plants showed severe symptoms. At the same time, the relative biomass of Rhizobium in each material was also consistent with its phenotype (FIG. 1C). This result indicates that RCR1 mediates A. thaliana resistance to clubroot.

### Example 2. Obtaining RCR1-Overexpressing B. napus and Analysis of its Resistance to P. brassicae

One. Obtaining RCR1 Overexpressing B. napus

### 1. Construction of RCR1 Gene Expression Vector

### 1) Obtaining Long Promoter RCR1 Gene Fragment

The DNA of A. thaliana ecotype Est-1 was extracted and performed PCR amplification using the primer RCR1-F-6k and the primer RCR1-R in Example 1 to obtain a PCR product of 7288 bp in size, and its nucleotide sequence is shown in SEQ ID NO: 5, termed p6kRCR1 gene, including a promoter sequence of approximately 6 kb, an encoding region of approximately 1.1 kb, and a UTR region. The primer sequences are as follows:

RCR1-F-6k:
(SEQ ID NO: 22)
5′-CGAATTCCGAATTCGAGCTCCCTAATCCGATCGAGTACAA-3′.

### 2) Obtaining RCR1 Gene Expression Vector

A recombinant vector pCAMBIA1300-p6kRCR1 was obtained by recombining p6kRCR1 into the pCAMBIA1300 vector backbone according to the method in step 2 of step one of Example 1, and then was sequenced.

The sequencing results show that the recombinant vector pCAMBIA1300-p6kRCR1 is a vector obtained by inserting the DNA molecule shown in SEQ ID NO: 5 into the KpnI restriction enzyme cutting site of the pCAMBIA1300 vector. In the DNA molecule shown in SEQ ID NO: 5, positions 1-6131 correspond to the promoter sequence, positions 6132-6578 correspond to the RCR1 genome sequence, and positions 6578-7288 correspond to the 3′UTR region.

2. Obtaining RCR1 Overexpressing B. napus

According to the method in step 3 of step one of Example 1, the recombinant vector pCAMBIA1300-p3.6kRCR1 was introduced into the clubroot-susceptible material Zhongshuang 11 (ZS11). The transgenic experiment was entrusted to Wuhan Boyuan Biotechnology Co., Ltd to complete. After obtaining the transgenic seedlings, the transgenic seedlings were screened according to the method of Example 1 to obtain the transgenic positive seedlings, and RCR1 overexpressing B. napus p3.6kRCR1_ZS11 was obtained.

According to the method in step 3 of step one of Example 1, the recombinant vector pCAMBIA1300-p6kRCR1 was introduced into clubroot-susceptible material Zhongshuang 11 (ZS11). The transgenic experiment was entrusted to Wuhan Boyuan Biotechnology Co., Ltd to complete. After obtaining the transgenic seedlings, the transgenic seedlings were screened according to the method of Example 1 to obtain RCR1-overexpressing B. napusp6kRCR1_ZS11 #1 and p6kRCR1_ZS11 #2.

Two. Analysis of Resistance of RCR1-Overexpressing B. napus to P. brassicae

According to the method in step two of Example 1, B. napus p3.6kRCR1 ZS11, p6kRCR1 ZS11 and ZS11 were overexpressed with RCR1 driven by promoters of different lengths and inoculated with P. brassicae PbDy or PbXm, where PbDy was P. brassicae from Dayi area, Sichuan and PbXm was from Xinmin area, Shenyang (identified as physiological race No. 4).

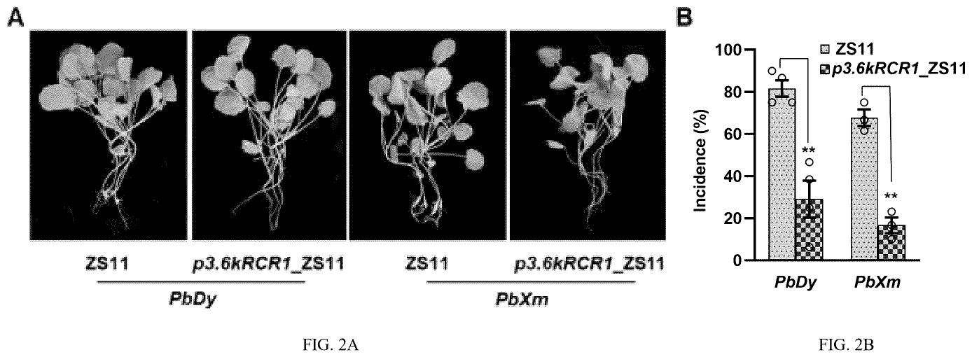

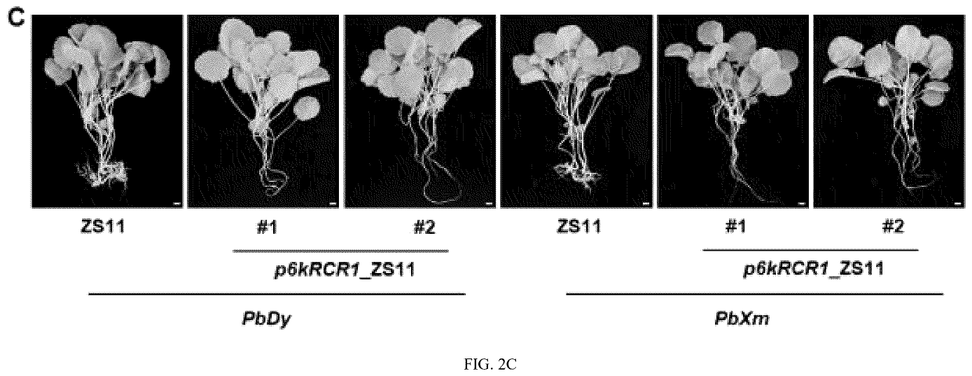

The plant phenotype was observed 21 days after inoculation, and it was found that ZS11 showed obvious susceptibility to two kinds of P. brassicae, PbDy and PbXm, that is, spindle-shaped tumors were formed in the roots of the plants, while RCR1 overexpressed B. napus p3.6kRCR1_ZS11 and p6kRCR1_ZS11 which were driven by promoters of different lengths were inoculated with the two bacteria, their underground parts developed normally (FIG. 2A). And when inoculated with P. brassicae from different regions, the incidence of RCR1 overexpression B. napus p3.6RCR1_ZS11 driven by the 3.6k promoter was significantly lower than that of ZS11 (FIG. 2B). It shows that the clubroot disease resistance gene RCR1 can be used to improve the B. napus clubroot disease resistance. It further illustrates that the RCR1 gene driven by the p3.6kRCR1 promoter is a key element in plant clubroot resistance, and its expression in other clubroot-susceptible plants can improve or restore plant clubroot resistance.

The above results show that the RCR1 gene and its promoter p3.6kRCR1 are related to plant clubroot resistance. The RCR1 gene and the RCR1 gene driven by the p3.6kRCR1 promoter can be used for the cultivation and identification of disease-resistant plant varieties in agricultural production, playing an important role in the field of plant disease-resistance breeding.

### INDUSTRIAL APPLICATION

The present invention demonstrates through experiments that when RCR1-transgenic A. thaliana or RCR1-transgenic B. napus is infected with P. brassicae, the resistance to P. brassicae is significantly enhanced as compared with wild-type A. thaliana or B. napus; and the resistance to P. brassicae of RCR1 gene-knock out A. thaliana is significantly reduced compared with the wild-type A. thaliana. It is indicated that the RCR1 gene is related to the resistance of the plant to P. brassicae, and the RCR1 protein and the coding gene thereof can be used for cultivating clubroot-resistant varieties in agricultural production. The present invention has great significance for cultivating disease-resistant varieties, especially for breeding new varieties of clubroot-resistant Brassicaceae crops, and can be used for cultivating and identifying disease-resistant plant varieties required by agricultural production.

What is claimed is:

1. A DNA molecule, comprising, in order from a 5′ end, a transcription regulatory element of a resistance to clubroot 1 (RCR1) gene and the RCR1 gene; wherein the transcription regulatory element of the RCR1 gene is the following A1) or A2):
A1) a first DNA molecule as shown at positions 1-3643 of SEQ ID NO: 3; or
A2) a second DNA molecule having 50% or higher identity to a nucleotide sequence of the first DNA molecule defined in A1) and having a transcriptional regulatory function;
and the RCR1 gene is the following B1) or B2):
B1) a third DNA molecule as shown at positions 3644-4090 of SEQ ID NO: 3; or
B2) a fourth DNA molecule having 50% or higher identity to a nucleotide sequence of the third DNA molecule defined in B1) and having a same function as the nucleotide sequence of the third DNA molecule defined in B1).

2. The DNA molecule according to claim 1, wherein the DNA molecule comprises in order from the 5′ end, the transcription regulatory element of the RCR1 gene, the RCR1 gene, and a 3′UTR region;
wherein a nucleotide sequence of the 3′UTR region is the following C1) or C2):
C1) a fifth DNA molecule as shown at positions 4091-4800 of SEQ ID NO: 3; or
C2) a sixth DNA molecule having 50% or higher identity to a nucleotide sequence of the fifth DNA molecule defined in C1) and having a same function as the nucleotide sequence of the fifth DNA molecule defined in C1).

3. The DNA molecule according to claim 1, wherein a nucleotide sequence of the DNA molecule is
as shown in SEQ ID NO: 3 or
has 50% or higher identity to SEQ ID NO: 3 and a same function as SEQ ID NO: 3.

4. A biological material related to the DNA molecule according to claim 1, wherein the biological material is a recombinant vector comprising the DNA molecule, a recombinant microorganism comprising the DNA molecule, or a transgenic plant cell line comprising the DNA molecule.

5. A method of using the DNA molecule according to claim 1 or a biological material related to the DNA molecule, comprising the following M1) or M2):
M1) regulating a disease resistance of a plant; or
M2) cultivating a transgenic plant or a gene-edited plant with an enhanced disease resistance;
wherein the biological material related to the DNA molecule is a recombinant vector comprising the DNA molecule, a recombinant microorganism comprising the DNA molecule, or a transgenic plant cell line comprising the DNA molecule.

6-8. (canceled)

9. The method according to claim 5, wherein the disease resistance is a resistance to Plasmodiophora brassicae.

10. The method according to claim 5, wherein the plant is as defined in M1) or M2) or M3) or M4):
M1) a monocotyledonous plant or a dicotyledonous plant;
M2) a Brassicaceae plant;
M3) Arabidopsis thaliana, or
M4) Brassica napus.

11. A method for cultivating a transgenic plant or a gene-edited plant with an enhanced disease resistance, comprising the following steps:
increasing a content and/or an activity of a RCR1 protein in a recipient plant to obtain the transgenic plant or the gene-edited plant, wherein a disease resistance of the transgenic plant or the gene-edited plant is higher than a disease resistance of the recipient plant;
wherein the RCR1 protein is as defined in N1) or N2) or N3) or N4):
N1) a protein having the amino acid sequence of SEQ ID NO: 2;
N2) a fusion protein obtained by attaching a tag to an N-terminus and/or a C-terminus of the protein of SEQ ID NO: 2;
N3) a plant disease resistance-related protein derived from the amino acid sequence of SEQ ID NO: 2 by a substitution, and/or a deletion, and/or an addition of one or more amino acid residues; or
N4) a plant disease resistance-related protein having 75% sequence identity to SEQ ID NO: 2.

12. The method according to claim 11, wherein the transgenic plant or the gene-edited plant having a higher disease resistance than the recipient plant reflects in one of the following X1) or X2):
X1) a lower disease incidence in the transgenic plant or the gene-edited plant compared to the recipient plant; or
X2) a lower disease index in the transgenic plant or the gene-edited plant compared to the recipient plant.

13. The method according to claim 11, wherein a method for increasing the content and/or the activity of the RCR1 protein in the recipient plant is to overexpress the RCR1 protein in the recipient plant.

14. The method according to claim 11, wherein the disease resistance of the transgenic plant or the gene-edited plant and the disease resistance of the recipient plant are a resistance to Plasmodiophora brassicae.

15. The method according to claim 11, wherein the recipient plant is as defined in M1) or M2) or M3) or M4):
M1) a monocotyledonous plant or a dicotyledonous plant;
M2) a Brassicaceae plant;
M3) Arabidopsis thaliana, or
M4) Brassica napus.

16. A transgenic plant or a gene-edited plant prepared by the method according to claim 11.

17. A method for cultivating a transgenic plant or a gene-edited plant with a reduced disease resistance, comprising steps of: reducing an activity and/or a content of a RCR1 protein in a recipient plant to obtain the transgenic plant or the gene-edited plant, wherein the transgenic plant or the gene-edited plant has a disease resistance lower than a disease resistance of the recipient plant;
the RCR1 protein is as defined in N1) or N2) or N3) or N4):
N1) a protein having the amino acid sequence of SEQ ID NO: 2;
N2) a fusion protein obtained by attaching a tag to an N-terminus and/or a C-terminus of the protein of SEQ ID NO: 2;
N3) a plant disease resistance-related protein derived from the amino acid sequence of SEQ ID NO: 2 by a substitution, and/or a deletion, and/or an addition of one or more amino acid residues; or
N4) a plant disease resistance-related protein having 75% sequence identity to SEQ ID NO: 2 and retaining a plant disease resistance-related activity.

18. The method according to claim 17, wherein the disease resistance of the transgenic plant or the gene-edited plant and the disease resistance of the recipient plant are a resistance to Plasmodiophora brassicae.

19. The method according to claim 17, wherein the recipient plant is as defined in M1) or M2) or M3) or M4):
M1) a monocotyledonous plant or a dicotyledonous plant;
M2) a Brassicaceae plant;
M3) Arabidopsis thaliana, or
M4) Brassica napus.

20. A transgenic plant or a gene-edited plant prepared by the method according to claim 17.
